(19) United States
(12) Patent Application Publication
(10) Pub. No.: US 2022/0061510 A1
(43) Pub. Date: Mar. 3, 2022

(54) Consumer Convenience Accessory

(71) Applicant: Andrew Spagnolo, Falls Church, VA (US)

(72) Inventor: Andrew Spagnolo, Falls Church, VA (US)

(21) Appl. No.: 17/224,216

(22) Filed: Apr. 7, 2021

Related U.S. Application Data
(60) Provisional application No. 63/073,476, filed on Sep. 2, 2020.

(51) Int. Cl. A45F 5/10; A61L 2/26; A61L 2/00

(52) U.S. Cl. CPC A45F 5/1026; A45F 2005/1093; A61L 2/0088; A61L 2/26

(57) ABSTRACT

This present invention relates to a consumer convenience accessory that enables an individual to comfortably yet manually transport a large number of items, such as carry bags, from one place to another. The accessory is a hand carrying device that is comprised of a handle portion and a lower arm and/or hooks that extend downwardly therefrom to support a plurality of carry bags each having a handle. In one embodiment, the carrying device may further comprise one or more repositionable arms to further secure the carry bags during transport and/or a hand sanitizer dispensing mechanism.

### CROSS-REFERENCE TO RELATED APPLICATION

The present application claims priority to, and the benefit of, U.S. Provisional Application No. 63/073,476, which was filed on Sep. 2, 2020 and is incorporated herein by reference in its entirety.

### FIELD OF THE INVENTION

The present invention relates generally to the field of consumer convenience accessories for a consumer. More specifically, the present invention relates to a carrying handle capable of supporting one or more heavy items simultaneously for transportation, such as from a shopping center to the vehicle of a consumer. In one embodiment, the novel carrying handle features a pair of generally U-shaped hooks connected to a C-shaped grip, wherein the hooks are useful for accommodating bags, propane tanks, paint cans, consumer products and numerous other types of items that may be carried via handles. Additionally, the novel carrying handle includes a plurality of notches on the C-shaped grip to support the various item to be carried, thereby enabling users to simply grasp the handle to carry multiple items simultaneously. The consumer convenience of the present invention saves extensive time and effort when transporting multiple items to and from a drop off or pick-up point. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, shopping, in one form or another, is a daily activity for most people. The activity of shopping typically involves a shopper using his or her hands to carry a plurality of shopping bags, totes, packages and the like, each of which may contain one or more purchased items and that may be individually or collectively heavy. Typically, the purchased items are kept in plastic bags, sacks, carrier bags, paper bags, retail bags, grocery bags or the like, which makes it easy to carry and transport the items from one place to another. However, carrying multiple bags in a single trip is oftentimes difficult and uncomfortable as the handles of the bags may cut into the hands of the individual transporting the items. Further, carrying multiple bags with heavy items with a single hand can cause strain and/or a sprain in the fingers, wrist, elbows, and shoulders of the individual. Additionally, depending upon the weight of the shopping bags and items being transported therein, there is the additional risk of lower back pain to the individual. In such scenarios, individuals may refrain from buying too many items in a single trip out of concern that the individual may not be able to carry everything to their destination.

Additionally, while carrying multiple traditional carry bags, people may lose their grip of the handles of the carry bags, thereby causing the items inside the carry bags to fall down on the floor leading to potential spoilage or breakage of the items. Without a proper grip, carrying multiple large items simultaneously may be extremely difficult or impossible depending on the weight of the items inside the carry bags. This may cause the individuals to have to make multiple trips to and from a location to transport several items, which is an inefficient use of the individual's time and a major inconvenience. Further, while carrying multiple heavy and bulky carry bags, an individual may lose his or her balance, particularly if walking on a slick or wet surface, which may result in the individual falling and becoming injured, not to mention the potential damage to the items being transported.

Therefore, there exists a long felt need in the art for a consumer convenience accessory device that allows an individual to carry multiple carry bags and other items in a single trip, thereby eliminating the need for the user to have to make multiple time-consuming trips to transport the items. There is also a long felt need in the art for an accessory device that enables an individual to conveniently carry multiple heavy and bulky carry bags without disproportionate weight shifting or undue strain on the individual's body, and that does not cause cuts in the hands of the individual from the handles on the carry bags. Moreover, there is a long felt need in the art for an accessory device that enables the individual to maintain a firm grip on multiple carry bags at the same time, and that reduces the likelihood of injury to the individual and/or damage to the items being carried. Finally, there is a long felt need in the art for an accessory device that is relatively inexpensive to manufacture and that is both safe and easy to use.

The subject matter disclosed and claimed herein, in one embodiment thereof, comprises a portable ergonomic handle for carrying a plurality of carry bags, sacks or the like simultaneously by their handles, and that includes an elongated grip bar having a grip along its bottom surface for engaging the fingers of the user. A connecting member extends downwardly (i.e., in the direction of the ground when in use) from the approximate center of the bottom surface of the grip bar and is comprised of a first end and a second end. The grip bar is positioned at the first end, and at least two generally U-shaped hooks are positioned at the second end. The two U-shaped hooks are preferably substantially similar in shape and size, but extend outwardly from the second end of the connecting member in opposite directions to one another. A plurality of carry bag handles can be supported by each of the generally U-shaped hooks, thereby enabling the user to transport a plurality of carry bags with a single hand and with less effort and greater stability than without the device, and without causing damage to the user's hands.

In an alternative embodiment, the consumer convenience accessory device may be comprised of a generally C-shaped structure, wherein the upper arm of the structure is supported by the user's hand and comprises a plurality of ridges and valleys to accommodate the user's fingers. The lower arm of the structure is preferably somewhat curved in shape and is capable of supporting a plurality of carry bags, wherein the lower arm may also comprise a plurality of notches or grooves to accommodate the various carry bag handles and more evenly distribute the weight of the items being transported by the device.

In this manner, the novel consumer convenience handle of the present invention accomplishes all of the forgoing objectives, and provides a relatively safe, convenient and cost-effective solution for carrying multiple carry bags with heavy and bulky items together in a single trip. The consumer convenience handle of the present invention is also user friendly, as it does not require the user to put much effort or labor in attempting to balance the different bags or sacks while transporting the carry bags with items from one location to another. Additionally, the one trip grip handle allows the user to maintain a firm grip while carrying bags, sacks or the like thereby preventing any injuries to the user's hands and other body parts.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

As stated above, the subject matter disclosed and claimed herein, in one embodiment thereof, comprises a consumer convenience accessory device for carrying a plurality of handled carry bags, sacks or the like simultaneously. The accessory device is preferably comprised of an integrally formed elongated grip bar, connecting member and two or more generally U-shaped hooks. More specifically, the elongated grip bar is comprised of a handle-like grip having a curved upper surface to accommodate the palm of the user's hand, and an undulated bottom surface for engaging the fingers of the user. A connecting member extends downwardly (i.e., in the direction of the ground when in use) from the approximate center of the bottom surface of the grip bar and is comprised of a first end and a second end, wherein the grip bar is positioned at the first end. Two or more generally U-shaped hooks, preferably substantially similar in shape and size, extend outwardly from the second end of the connecting member in opposite directions to one another to provide a degree of balance when in use. A plurality of carry bag handles can be supported by each of the generally U-shaped hooks, thereby enabling the user to transport a plurality of carry bags with a single hand and with less effort and greater stability than without the device, and without causing damage to the user's hands.

In an alternative embodiment, the consumer convenience accessory device may be comprised of a generally C-shaped structure. The C-shaped structure is preferably integrally formed and comprised of an upper arm and a lower arm, wherein the upper arm of the structure is supported by the user's hand and comprises a plurality of ridges and valleys to accommodate the user's fingers and a curved upper surface to accommodate the palm of the user. The lower arm of the C-shaped structure is also preferably somewhat curved in shape and is capable of supporting a plurality of carry bags, wherein the lower arm may also comprise a plurality of notches or grooves to accommodate the various carry bag handles and more evenly distribute the weight of the items being transported by the device. The curvature of the lower arm also ensures that the plurality of carry bags do not slip off of the device when in use.

In yet a further embodiment of the present invention, any of the embodiments of the accessory device described herein may further be comprised of a durable plastic, metal, wood or elastomeric material, provided that the same is flexible enough to allow for some bending but rigid enough to support at least 50 lbs. of weight. As previously stated, the accessory device is preferably integrally formed, such as through injection molding, blow molding or the like, and may also be impregnated or coated with an anti-microbial agent to kill any bacteria, germs, diseases, microbes or the like that may be present on the carry bag handles. Additionally, or alternatively, the accessory device may also comprise a reservoir capable of retaining and dispensing an anti-microbial agent, such as hand sanitizer on the user's hands to kill any bacteria, germs, diseases, microbes or the like that the user may come into contact with while shopping.

In yet a further embodiment of the present invention, the accessory device may comprise one or more repositionable clips that can be placed over the carry bag handles while the same are being supported by the device to further secure the same to the device and prevent slippage that could cause damage to the items being transported in the carry bags. In one embodiment, the one or more repositionable clips may be spring-loaded to avoid the unintentional repositioning of the same when the device is in use.

The consumer convenience handle device of the present invention is particularly advantageous because it provides a user with the capacity to carry multiple carry bags in a secure manner and in a single trip. Further, the individual can double said capacity by positioning one handle device of the present invention in each hand, which will also help to provide balance to the individual transporting the plurality of carry bags. An individual may use the handle device to carry virtually any object with a handle including, without limitation, carry bags, propane tanks, paint cans, consumer goods and numerous other types of items with flexible or rigid handles. The handle device reduces the number of trips typically associated with transporting multiple items and does not cause damage to the user's hands. Further, because of the unique design of the handle device, the carry bag handles do not move or slide on the surface of the handle when hung in the notches, grooves, or U-shaped hooks.

In use, the user would simply place the carry bag handles onto the notches or U-shaped hooks, ideally balancing the weight relatively evenly across the same or across two handle devices if one will be supported by each hand of the user. The user will then use the ergonomical handle portion of the carrying device to transport the device and the plurality of carry bags or other items being transported thereon to the desired destination. Once at the destination, the user simply rests the bags on a surface and unhooks each of the carry handles from the notches or generally U-shaped hooks. Further, if the carry handles contain hand sanitizer therein, the user may apply the same to his or her hands to kill any bacteria, germs, diseases, microbes or the like that the user may come into contact with while shopping or from handling the carry bags.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1A illustrates a perspective view of one potential embodiment of the convenience accessory device of the present invention in accordance with the disclosed architecture, wherein the device is being used to transport a plurality of carry bags;

FIG. 1B illustrates a perspective view of an alternative potential embodiment of the convenience accessory device of the present invention in accordance with the disclosed architecture, wherein the device is comprised of three evenly spaced-apart hooks;

FIG. 2 illustrates a perspective view of a user using the convenience accessory device of FIG. 1A to transport a plurality of carry bags in accordance with the disclosed architecture;

FIG. 3 illustrates a perspective view of an alternative potential embodiment of the convenience accessory device of the present invention in accordance with the disclosed architecture, wherein the device is being used to transport a plurality of carry bags;

FIG. 4 illustrates a perspective view of a user using the convenience accessory device of FIG. 3 to transport a plurality of carry bags in accordance with the disclosed architecture;

FIG. 5 illustrates a partial cross sectional view of the handle portion of one potential embodiment of the convenience accessory device of the present invention in accordance with the disclosed architecture, wherein the padding and internal reservoir of hand sanitizer is clearly shown;

FIG. 6 illustrates a partial perspective view of the bottom portion of one potential embodiment of the convenience accessory device of the present invention in accordance with the disclosed architecture, wherein the repositionable clips for holding the carry bag handles in place during use are clearly shown; and

FIG. 7 illustrates a illustrates a perspective view of an alternative embodiment of the convenience accessory device of the present invention in accordance with the disclosed architecture.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a consumer convenience accessory device that allows an individual to carry multiple carry bags and other items in a single trip, thereby eliminating the need for the user to have to make multiple time-consuming trips to transport said items. There is also a long felt need in the art for an accessory device having an antimicrobial coating thereon, and that is capable of dispensing hand sanitizer to prevent the transmission of bacteria, germs, diseases, microbes and the like. Moreover, there is a long felt need in the art for an accessory device that enables an individual to conveniently carry multiple heavy and bulky carry bags without disproportionate weight shifting or undue strain on the individual's body, and that does not cause cuts in the hands of the individual from the handles on the carry bags. Additionally, there is a long felt need in the art for an accessory device that enables the individual to maintain a firm grip on multiple carry bags at the same time, and that reduces the likelihood of injury to the individual and/or damage to the items being carried. Finally, there is a long felt need in the art for an accessory device that is relatively inexpensive to manufacture and that is both safe and easy to use.

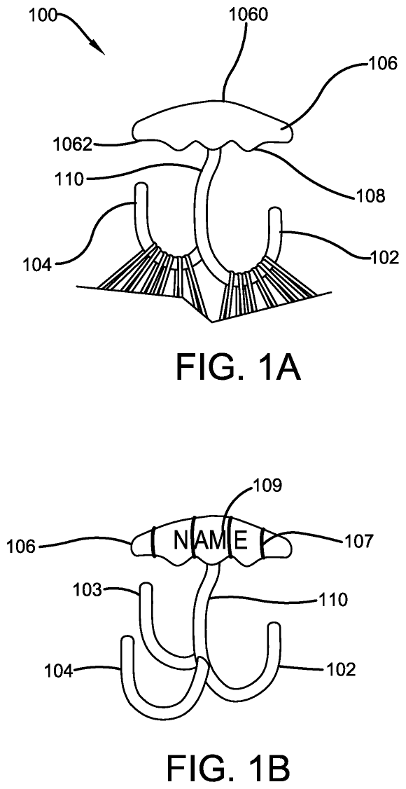

Referring initially to the drawings, FIG. 1A illustrates a perspective view of one potential embodiment of the convenience accessory device 100 of the present invention in accordance with the disclosed architecture, wherein the device 100 is being used to transport a plurality of carry bags. More specifically, the device 100 is comprised of an integrally formed grip bar 106, a connecting member 110 and two generally U-shaped hooks 102, 104 to support and transport the items, such as a plurality of carry bags, from place to place. The generally U-shaped hooks 102, 104 are connected to one end of the connecting member 110 which is, in turn, connected to the lower surface 1062 of the handle or gripping bar 106 at its opposite end. The connecting member 110 is preferably centrally disposed on the lower surface 1062 of the handle 106 and extends generally downwardly (i.e., in the direction of the ground when in use) from the handle 106. The handle 106 is further comprised of a curved upper surface 1060 to ergonomically fit within the palm of the user, and the lower surface 1062 of the handle 106 is preferably comprised of a grip 108 comprising a plurality of alternating ridges and valleys to accommodate the user's fingers when holding the device 100.

As noted above, the connecting member 110 is preferably connected at the approximate center of the handle 106 to give proper balance to the device 100, and the generally U-shaped hooks 102, 104 are of substantially similar curvature and size as to allow the user to carry multiple items simultaneously. The device 100 may be made of any suitable material such as, but not limited to, a durable metal (e.g., zinc, aluminum, steel, iron, brass, etc.), plastic, carbon composite or any other lightweight material. If a metal is used, the same should have rust resistant properties or be powder coated or painted. Further, the device 100 can be of any color to suit the user's needs and or preferences, and may additionally comprise a logo, indicia, personalized name, caption, slogan, or the like printed on the body of the device 100. For example, the handle 106 may contain the name 109 (see e.g., FIG. 1A) of the retail establishment that provided the device as part of a promotional or loyalty giveaway to its customers. The length of the handle portion 106 should be large enough to fit the human hand and preferably ranges from about 3 inches to about 6 inches.

Each generally U-shaped hook 102, 104 should extend outwardly in opposite directions to provide greater balance to the device 100, and should also be capable of safely supporting approximately 25 pounds of weight. However, the capacity of the U-shaped hooks 102, 104 is not so limited, and one or both of the U-shaped hooks 102, 104 can be designed to accommodate additional weight to suit the wants and needs of the user.

FIG. 1B illustrates a perspective view of an alternative potential embodiment of the convenience accessory device 100 of the present invention in accordance with the disclosed architecture, wherein the device 100 is comprised of three evenly spaced apart hooks 102, 103, 104, 106. More specifically, each of the generally U-shaped hooks 102, 103, 104 should be separated from one another by approximately 120 degrees so as to more equally distribute the weight being transported by the device 100, and to provide better balance to the user. In addition, the exterior surface of the handle portion 106 may further comprise one or more ridges or grips 107 to provide an anti-slip surface and additional comfort to the user. Marketing or personalization indicia 109 may also be provided on the exterior surface of the handle 106 to denote, for example, the name of the retailer, owner, sports team or the like.

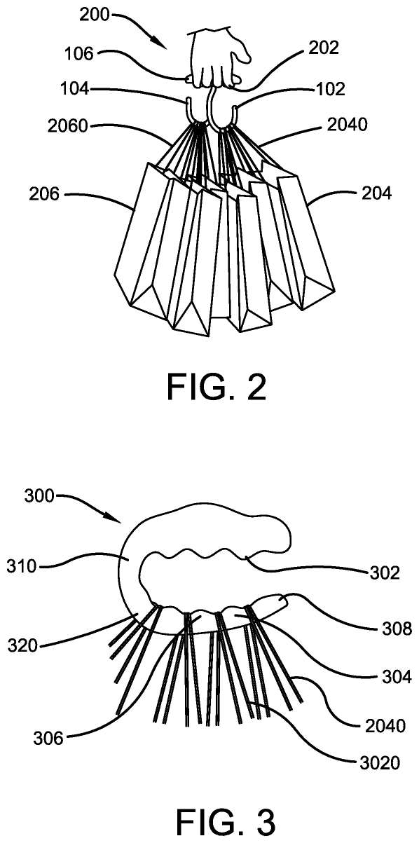

FIG. 2 illustrates a perspective view of a user 200 using the convenience accessory device 100 of FIG. 1A to transport a plurality of carry bags 204, 206 in accordance with the disclosed architecture. More specifically, the user 200 holds the carrying device 100 by gripping the grip handle 106 in his or her hand 202, and the carry bags 204, 206 are supported or hung on the generally U-shaped hooks 102, 104 via the bag handles 2040, 2060. Importantly, the generally U-shaped hooks 102, 104 are designed so that the carry bags 204, 206 remain secured within the hooks 102, 104 (and possibly 103 of the embodiment depicted in FIG. 1B is utilized) and do not slip off of the device 100 wherein the contents of the carry bags 204, 206 could become damaged.

FIG. 3 illustrates a perspective view of an alternative potential embodiment of the convenience accessory device 300 of the present invention in accordance with the disclosed architecture, wherein the device 300 is being used to transport a plurality of carry bags. More specifically, the device is comprised of a generally C-shaped structure that is preferably integrally formed and comprised of an upper arm 310 and a lower arm 320, wherein the upper arm 310 of the device 300 may be supported by a user's hand and comprises a grip 302 comprised of a plurality of ridges and valleys to accommodate the user's fingers and a curved upper surface to accommodate the palm of the user.

The lower arm 320 of the C-shaped structure 300 is also preferably somewhat curved in shape and is capable of supporting a plurality of carry bags, wherein the lower arm 320 may also comprise a plurality of notches or grooves 304, 306, 308 to accommodate the various carry bag handles and more evenly distribute the weight of the items being transported by the device 300. More specifically, the notches or grooves 304, 306, 308 provide a recess in between every two notches where the bag handles 2040 may be supported and hung. For example, the bag handles 2040, 3020 may be placed between the notches 304, 308. The notches give a secure place to position the bag handles so that the handles do not slip or move while carrying the same. Further, the curvature of the lower arm 320 also ensures that the plurality of carry bags do not slip off of the device 300 when in use.

Similar to device 100, both the upper arm 310 and the lower arm 320 are made from a durable material such as, but not limited to, a metal (e.g., zinc, aluminum, steel, iron, brass, etc.), plastic, carbon composite or any other lightweight material. If a metal is used, the same should have rust resistant properties or be powder coated or painted. Further, the device 300 can be of any color to suit the user's needs and or preferences, and may additionally comprise a logo, indicia, personalized name, caption, slogan or the like printed on the body of the device 300.

The grip 302 is a comfortable grip with which to hold the handle device 300, wherein the user's fingers are comfortably placed with proper alignment along the grip 302 so that a relatively heavy load can easily be transported and balanced via the device 300. More specifically, the grip 302 has protrusions acting as finger alignment bumps extending below for finger alignment. The grip 302 may further comprise a foam or cushion padding (see e.g., FIG. 5), and both the upper arm 310 and the lower arm 320 are preferably generally cylindrical in nature and have a thickness of approximately one quarter of an inch. Additionally, the gap between the upper arm 310 and the bottom arm 320 is such that the fingers of the user holding the handle device 300 are easily and comfortably placed and do not rub against the bag handles, which could lead to breakage of said bags and damage to the goods being transported therein. Finally, a spring (not shown) may also be present in the curved portion connecting the upper and lower arm portions 310, 320 to maintain the shape of the handle device 300 and prevent deformation from occurring.

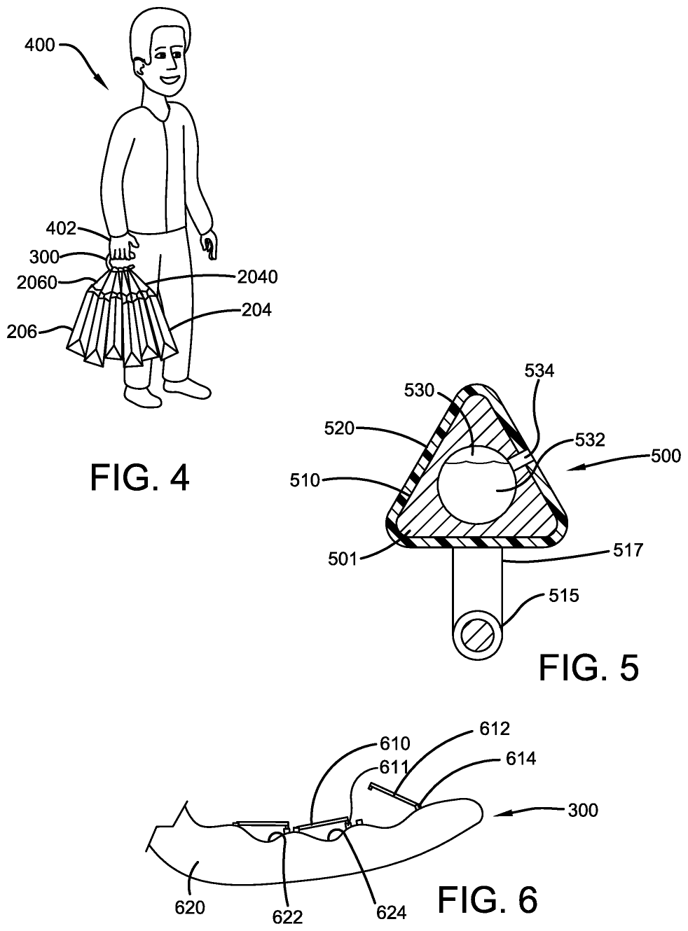

FIG. 4 illustrates a perspective view of a user 400 using the convenience accessory device 300 of FIG. 3 to transport a plurality of carry bags in accordance with the disclosed architecture. More specifically, the user 400 holds the generally C-shaped device 300 using one of his or her hands 402 by holding the grip 302 present on the upper arm 310 of the device 300. The bags 204, 206 are then hung by their handles 2040, 2060, respectively, on the notches present on the lower arm 320. In this manner, the user 400 can comfortably and painlessly carry a plurality of carry bags in a single trip. The grip 320 of the handle 300 may be comprised of one or more of a neoprene, foam, natural rubber, synthetic rubber, silicone rubber, phenolic material, leather, light plastic or combination thereof.

FIG. 5 illustrates a partial cross sectional view of the handle portion of one potential embodiment of the convenience accessory device 100, 300 of the present invention in accordance with the disclosed architecture, wherein the padding 520 and internal reservoir 530 of hand sanitizer 532 is clearly shown. More specifically, the carrying handle 500 may have a roughly triangular shape 510, the base 501 of which is sized and configured to accommodate the proximal phalange of each of the user's fingers and allow for the natural curvature of the fingers around the handle 500. The handle 500 is covered with a compressible foam padding 520 to add comfort to the user and allow the user to have a more secure grip. The connecting member 515 is also provided with a compressible foam 517 which extends at least a portion of the way along the connecting member 515 so that when the handle 500 is placed in the user's hand and the connecting member 515 is between the user's fingers the additional compressible foam 515 prevents the connecting member 515 from rubbing on the fingers and causing irritation. The compressible foam padding 517, 520 may be a silicone foam, polyethylene foam, polyurethane, EPDM, neoprene, urethane or any combination thereof.

Reservoir 530 is a void or cavity within the device 100, 300 that can be used to store and dispense hand sanitizer 532 through an opening 534 that extends between the reservoir 530 and the exterior surface of the handle device 100, 300. The user may apply the hand sanitizer 532 to his or her hands to kill any bacteria, germs, diseases, microbes or the like that the user may come into contact with while shopping or from handling the carry bags.

FIG. 6 illustrates a partial perspective view of the bottom portion of one potential embodiment of the convenience accessory device 100, 300 of the present invention in accordance with the disclosed architecture, wherein the repositionable clips 610, 620 for holding the carry bag handles in place during use are clearly shown. More specifically, the lower arm 620 of the device may be comprised of two or more notches 622, 624 that have repositionable locking elements or arms 610, 612. The locking elements 610, 612 include a base 611 into which the locking elements or arms may be secured in position over the notches 622 and 624. Each of the arms 610, 612 are pivotable about a hinge 614. Through use of the locking arms 610, 612, once the bag handles are disposed in the notches 622, 624, the arms 610, 612 prevent the carry bag handles from coming out of the notches 622, 624 which may otherwise occur through the normal movement or swaying of an individual when carrying the bags to their destination.

As stated above, each of the devices 100, 300 of the present invention may also be made from a plastic or other material into which anti-microbial materials (not shown) may be incorporated. Alternatively, an anti-microbial coating such as, but not limited to, Microban® may be applied to the surface of the same.

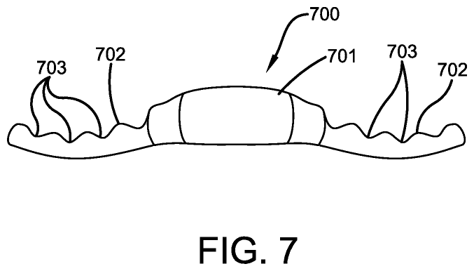

FIG. 7 illustrates a illustrates a perspective view of an alternative embodiment of the convenience accessory device 700 of the present invention in accordance with the disclosed architecture. The convenience accessory device 700 is preferably comprised of an elongated and curved member having a handle portion 701 positioned between two end portions 702, wherein each of the end portions 702 have a plurality of notches 703 therein for engaging, for example, propane tank handles. More specifically, a propane tank (not shown) can be positioned on each end portion 702 and the user can easily use the device to transport a pair of tanks with a single hand via the handle 701 of the convenience accessory device 700.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “handle apparatus”, “bar handle with two U-shaped hooks”, and “bar handle”, are interchangeable and refer to the bar handle with two U-shaped hooks 100 of the present invention. Similarly, as used herein “one trip C bar” and “C-shaped handle”, are interchangeable and refer to the handle devices 100, 300 of the present invention.

Notwithstanding the forgoing, all embodiments of the handle devices 100, 300 of the present invention can be of any suitable size and configuration as is known in the art without affecting the overall concept of the invention, provided that they accomplish the above stated objectives. One of ordinary skill in the art will appreciate that the size, configuration and material of the handle devices 100, 300 as shown in the FIGS. are for illustrative purposes only, and that many other sizes and shapes of the handle devices 100, 300 and their various components are well within the scope of the present disclosure. Although the dimensions of the handle devices 100, 300 are important design parameters for user convenience, the handle devices 100, 300 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A consumer convenience accessory comprising:
a gripping handle sized and configured to fit within a hand of a user;
a connecting portion having a first end attached to the gripping handle and a second end; and
a bag holding portion attached to the second end of connecting portion.

2. The consumer convenience accessory as recited in claim 1, wherein the bag holding portion is comprised of a plurality of notches.

3. The consumer convenience accessory as recited in claim 2, wherein at least one of the plurality of notches comprises a repositionable locking arm covering the at least one of the plurality of notches when the repositionable locking arm is in a closed position.

4. The consumer convenience accessory as recited in claim 1, wherein the bag holding portion is comprised of least two hook portions.

5. The consumer convenience accessory as recited in claim 4, wherein each of the at least two hook portions are approximately the same size and the at least two hook portions are directed in opposite directions from one another.

6. The consumer convenience accessory as recited in claim 1, wherein the bag holding portion is comprised of three hook portions.

7. The consumer convenience accessory as recited in claim 6, wherein each of the three hook portions are spaced apart from the remaining hook portions by approximately 120 degrees.

8. The consumer convenience accessory as recited in claim 1 further comprising an anti-microbial material.

9. The consumer convenience accessory as recited in claim 8, wherein the anti-microbial material is impregnated into the consumer convenience accessory.

10. The consumer convenience accessory as recited in claim 1, wherein the gripping handle further comprises a compressible foam.

11. The consumer convenience accessory as recited in claim 10, wherein the compressible foam is selected from a silicone foam, a polyethylene foam, a polyurethane, a EPDM, a neoprene, a urethane or a combination thereof.

12. The consumer convenience accessory as recited in claim 1 further comprising an internal reservoir and a continuous opening extending between the internal reservoir and an exterior surface of the consumer convenience accessory.

13. The consumer convenience accessory as recited in claim 12 further comprising a supply of a hand sanitizer positioned within the internal reservoir.

14. A C-shaped bag carrier comprising:
a handle portion sized and configured to fit within a human hand and having a length from about 3 inches to 6 inches, wherein a bottom surface of the handle portion comprises a plurality of finger recesses;
a connecting portion having a first end and a second end, wherein the first end is integrally formed with the handle portion; and
a carrying portion integrally formed with the second end and comprising a plurality of notches positioned on its top surface.

15. The C-shaped bag carrier as recited in claim 14, wherein, the handle portion comprises a compressible foam material.

16. The C-shaped bag carrier as recited in claim 15, wherein the compressible foam material is selected from a silicone foam, a polyethylene foam, a polyurethane, a EPDM, a neoprene, a urethane or a combination thereof.

17. The C shaped bag carrier as recited in claim 14 further comprising a reservoir containing a supply of an anti-microbial agent.

18. A carrier device for consumer goods comprising:
a grip bar comprising a compressible foam;
a connecting member attached to a central location of the grip bar on a first end of the connecting member and extending outwardly therefrom; and
at least two hooks attached to a second end of the connecting member, wherein each of the two hooks extend outwardly in opposite directions.

19. The carrier device for consumer goods as recited in claim 18, wherein the compressible foam is selected from a silicone foam, a polyethylene foam, a polyurethane, a EPDM, a neoprene, a urethane or a combination thereof.

20. The carrier device for consumer goods as recited in claim 18 further comprising a reservoir containing a supply of an anti-microbial agent.
